(19) United States
(12) Patent Application Publication
(10) Pub. No.: US 2026/0007817 A1
(43) Pub. Date: Jan. 8, 2026

(54) INFUSION PUMP HOUSING WITH A SECURING DEVICE TO PREVENT REMOVAL FROM AN INFUSION POLE

(71) Applicant: B. Braun Melsungen AG, Melsungen (DE)

(72) Inventor: Marcel Stich, Nieste (DE)

(21) Appl. No.: 19/249,614

(22) Filed: Jun. 25, 2025

(30) Foreign Application Priority Data
Jul. 2, 2024 (EP) 24185926.3

(51) Int. Cl. A61M 5/14

(52) U.S. Cl. CPC A61M 5/1414

(57) ABSTRACT

A lockable housing accommodates at least a section of an infusion pump, preferably a syringe pump. The housing has a flap, in particular a front flap, which can be locked by a locking device. The flap can be fixed to a main section of the housing in a closed position. The housing can be secured to an infusion pole by a securing device. The securing device can be moved from an unsecured or released position into a secured position. The securing device can be fixed in the secured position and locked by the locking device of the flap.

### CROSS-REFERENCE TO RELATED APPLICATION

This application claims priority under 35 U.S.C. § 119 to European Application No. 24185926.3, filed on July 2, 2024, the content of which is incorporated by reference herein in its entirety.

### FIELD

The present disclosure relates to a securing device for an infusion pump, e.g., a syringe pump, to prevent the removal of an infusion pole or a pole or stand.

### BACKGROUND

In intensive care units of hospitals, infusion pumps, e.g., syringe pumps, are mounted on infusion poles and then dismantled again after a course of therapy. The infusion pole is also called a pole or a tripod. For this purpose, clamping devices are known which, on the one hand, are fastened to the back of the infusion pump's housing and, on the other hand, can be releasably clamped to the infusion pole.

To secure such infusion pumps against unauthorized disassembly and removal from the infusion pole, cable loops are known that are similar to a laptop lock. These have to be threaded with great effort.

The notifying party itself markets a lockable housing for a syringe pump known as a "lockbox." The housing completely encloses the affected syringe pump and has a front flap that can be locked with a lock, whereby the front flap is fixed in a closed position on a main section of the housing. These types of housings are used, for example, in patient-controlled analgesia, in which the patient can determine the amount, e.g., of a painkiller themselves.

In order to secure the syringe pump against unauthorized disassembly and removal from the infusion pole, the housing-or more precisely, its main section-can be secured to the infusion pole by means of a detachable and lockable securing device. The securing device has a latch that can be pivoted behind the infusion pole and locked with a lock so that the housing and the latch enclose the infusion pole on all sides.

The disadvantage of the securing device of such housings is that they require an extra lock, whereby the housing has a lock for the front flap and a lock for the latch of the securing device. This means that medical personnel have to operate two locks to secure the affected infusion pump. This disadvantage is multiplied accordingly when several infusion pumps have to be fastened and secured to a single infusion pole for a single patient.

### SUMMARY

The object of the present disclosure is to provide a lockable housing that is designed to enclose an infusion pump by means of a flap, for example a front flap, and with a lockable securing device for securing to an infusion pole, wherein a lock is to be saved or omitted.

The lockable housing according to the disclosure can also be called a lockbox and is designed and prepared for at least partial accommodation and locking of an infusion pump, preferably a syringe pump. For this purpose, the housing has a flap, in particular a front flap, which can be locked by means of a locking device, in particular a lock with a key, whereby the (front) flap can be fixed in a closed position on a main section of the housing. Furthermore, the main section of the housing can be secured to an infusion pole, which may also be called a pole, or to a tripod by means of a (detachable and lockable) securing device. The securing device can be moved, for example, translationally or rotationally, in particular rotationally, from an unsecured or released position into a secured position. The securing device can be fixed in the secured position by means of the (front) flap locking device and (indirectly) locked.

The securing device can be pulled out in a translational direction from the released to the secured position under the infusion pump and can be fixed behind the housing on the infusion pole. However, it is preferred if the securing device can be moved rotationally between the released position and the secured position (bidirectionally), in particular if it can be pivoted.

In a preferred exemplary embodiment, the securing device has a main body and a pivot joint by which the main body is coupled to the main section of the housing. The main body of the securing device can be pivoted in both directions between the released position and the secured position by means of the pivot joint. The main body has a securing section (e.g., a bar) that can be fixed to the (front) flap of the housing in the secured position.

At a region opposite the securing section with respect to the pivot joint, the main body may have a hook that is designed and arranged to engage behind an infusion pole. For this purpose, the hook has a preferably arcuate contact region for the infusion pole.

With such a securing device, it is possible to fix the affected housing to the rear infusion pole by means of a pivoting movement and, at the same time, to move the securing section to the opposite front region of the (front) flap, where it can be fixed, using the same pivoting movement.

In a simple design from a manufacturing point of view, the main body of the securing device is flat, e.g., a stamped sheet metal part, on which the securing section and, if necessary, the hook are formed in one piece. This means that the main body can also be described as a contour lever.

In order to bridge a certain expansion of the housing, or more precisely a distance between the infusion pole and the (front) flap, it is preferred if a securing lever extends from the pivot joint, with the securing section formed at the end section, and/or if the hook is connected to the pivot joint via a preferably angled arm.

The securing device is preferably fixable in the secured position via its securing section preferably in a recess in the (front) flap, in a form-fitting manner. This fixes the hook in the infusion pole and the housing is secured to the infusion pump on the infusion pole to prevent removal.

When the securing device is designed as a pivotable main body, it can preferably be locked in the secured position by means of the locking device of the (front) flap indirectly in the following way: First, the main body is pivoted into the secured position, whereby the main body engages the infusion pole with its hook, for example, and at the same time the securing section comes into the region of the (front) flap. Then the (front) flap is closed, whereby the securing section is inserted into the recess of the (front) flap and fixed opposite the (front) flap. Then the (front) flap is locked with its locking device, which indirectly also fixes the securing device and thus locks it.

In a preferred embodiment of the indirect lockability, a pivot joint is arranged on an initial edge of the (front) flap, via which the (front) flap is hinged to the main section of the housing. The recess is preferably formed on a second edge of the (front) flap, opposite the first edge. The recess is further adapted to move over or onto the securing section when the (front) flap is pivoted into its closed position.

When the housing with the infusion pump is installed on the infusion pole, the first edge is preferably an upper edge, while the second edge is a lower edge. The recess can be moved over or onto the securing section when the (front) flap is folded down in its closed position.

If the main body is flat, e.g., a stamped sheet metal part, it is particularly preferred if the main body is coupled to a bottom of the main section of the housing via the pivot joint. In this case, a pivot axis of the pivot joint is perpendicular to the main body and perpendicular to the bottom, and the main body and the bottom are parallel to each other. This saves a lot of installation space, or to be more precise, the housing can be designed to be small. The main body and the bottom are horizontal when the housing is installed.

To prevent the main body and in particular, the hook, from swinging around uncontrollably, even in the released position and in particular when the housing is removed from the infusion pole, a latching device for the securing section is preferably provided on the bottom.

When the angle between the secured position and the released position of the main body is between 45 and 135 degrees, preferably around 90 degrees, the arm can be parked in the released position in a space-saving way and the hook can be placed (parked) as close as possible to the housing.

Preferably, in the secured position of the main body, its securing lever, at the end of which the securing section is formed, extends radially in the direction away from the pivot joint towards the recess of the (front) flap.

In the released position of the main body, the securing lever extends preferably radially in the direction away from the pivot joint towards the latching device, with the securing section engaging in the latching device.

In accordance with the state of the art, the infusion pump is accommodated in the housing, and the housing can be attached to the infusion pole by means of a detachable fastening device. This allows the infusion pump to be indirectly attached to the infusion pole.

In contrast to this, an infusion pump arrangement with an infusion pump is also disclosed in this document, which can be attached, or the housing of which can be attached, directly to an infusion pole by means of a releasable fastening device, preferably by means of a clamping device. The infusion pump is accommodated at least in sections in a housing according to the disclosure.

The infusion pump thus carries the housing in accordance with the disclosure, wherein the housing carries the securing device, which in turn engages directly behind the infusion pole in its secured position.

In the context of the infusion pump assembly comprising an infusion pump and a clamping device fastened thereto and the housing carried by the infusion pump, the infusion pump is particularly well secured against removal if the clamping device has two clamping jaws that can be moved radially towards each other, and if the hook of the fastening device engages behind the infusion pole on a side opposite the housing or facing away from the housing in the secured position. In other words, the infusion pump is particularly well secured against removal when, viewed in the circumferential direction of the infusion pole, one of the clamping jaws is positioned first, then the contact region of the hook, then the other clamping jaw, and then the rear side of the infusion pump or the housing are arranged. With this design, the housing with the infusion pump cannot be pulled off the infusion pole even after the clamping device has been opened, i.e., after the two clamping jaws have been moved apart.

### BRIEF DESCRIPTION OF THE DRAWINGS

FIG. 1 is an infusion pump assembly with an infusion pump and a housing according to one exemplary embodiment of the present disclosure in a bottom view perspective;

FIG. 2 is the infusion pump assembly from FIG. 1 in a bottom view, wherein the bottom of the housing is omitted;

FIG. 3 is a further representation of the infusion pump assembly from FIG. 1 in a bottom view, wherein the bottom of the housing is omitted; and

FIG. 4 is a further representation of the exemplary embodiment of the infusion pump assembly shown in FIG. 1, in a bottom view perspective.

### DETAILED DESCRIPTION

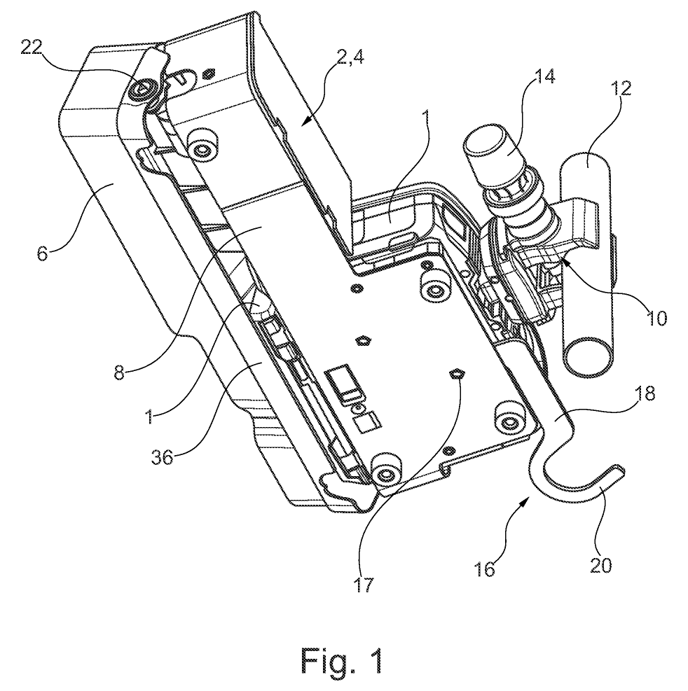

An exemplary embodiment of the present disclosure is described below, based on the associated figures. FIG. 1 shows an infusion pump assembly with an infusion pump 1 and a housing 2. The housing 2 has a main section 4 and a front flap 6 that can be folded and pivoted opposite it. For this purpose, the front flap 6 is hinged to the main section 4 at its first (upper, not visible) edge. The bottom 8 of the main section 4 can be seen in particular.

In the exemplary embodiment shown, the infusion pump 1 is a syringe pump that is clamped directly to an infusion pole 12, which is only shown in sections and is located next to a patient's bed, by means of a fastening device 10 designed as a clamping device. For this purpose, the housing 2 has a rear wall-free region through which the fastening device 10 extends. The fastening device 10 has two clamping jaws that are tightened against each other by means of a rotatable handle 14 and clamp the infusion pole 12 between them.

The housing 2 is carried by the infusion pump 1 and serves to protect the infusion pump 1 against unauthorized manipulation and unauthorized removal. For this purpose, the housing 2 can be closed completely or in sections at the front side (left side in FIG. 1), where the control elements and the syringe are located, using the front flap 6. To do this, the front flap 6 is folded down completely from the half-opened intermediate position shown in FIG. 1.

Furthermore, FIG. 1 shows a lock 22 for a (not shown) key, which is arranged on a lateral edge of the front flap 6 and which can be engaged with the edge of the main section 4 of the housing 2. The special feature of this lock 22 is explained in more detail below.

A securing device according to the disclosure consists of a pivot joint 17 and a flat main body 16. The main body 16 is shown in FIG. 1 in its unsecured and released position, in which it releases the housing and thus the infusion pump 1. In this case, the main body 16 is arranged partly between the infusion pump 1 and the bottom 8 of the main section 4 and partly outside it.

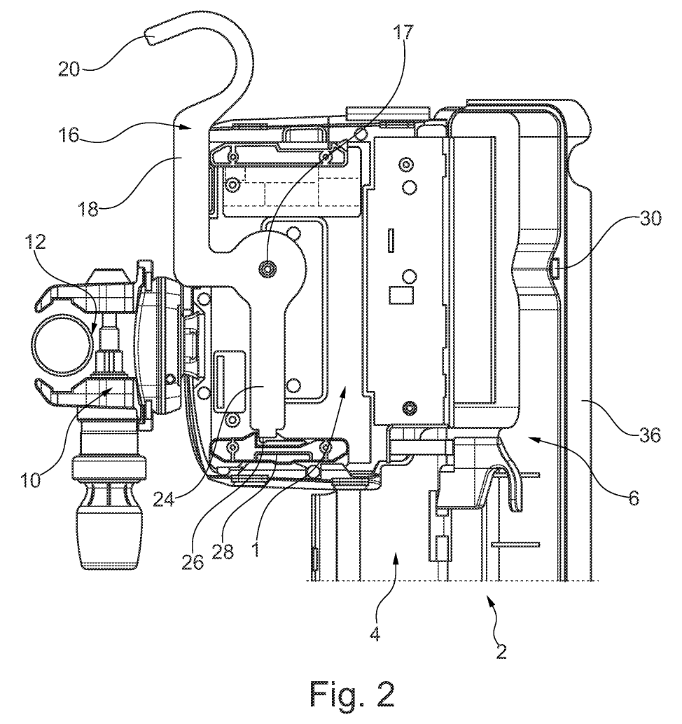

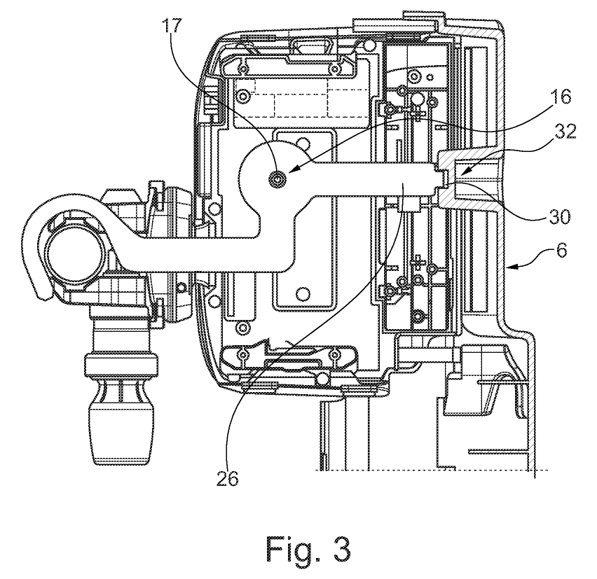

More specifically, in the released position of the securing device or its main body 16, as shown in FIG. 1, a securing lever (shown in FIGS. 2 and 3) having a securing section is completely located between the bottom 8 of the main section 4 and the infusion pump 1, while an arm 18 having a hook 20 is located in its parking position spaced from the infusion pole 12 and is therefore visible.

FIG. 2 is a section of the exemplary embodiment of the infusion pump assembly shown in FIG. 1, in a bottom view, with the bottom 8 of the main section 4 of the housing 2 removed. Unlike in FIG. 1, the front flap 6 is shown in its fully open position. In accordance with FIG. 1, the securing device or its main body 16 is shown in the released position.

It can be seen that the arm 18, which extends from the pivot joint 17 to the hook 20, is angled. In particular, it can be seen that a securing lever 24 extends radially from the pivot joint 17 in a region opposite the arm 18 with the hook 20, and a securing section 26 is formed on the free end section of the lever facing away from the pivot joint 17.

The two clamping jaws can be moved radially towards each other. In the secured position shown, the hook 20 engages behind the infusion pole 12 on one side (left in FIG. 2), which is opposite the housing 2. The infusion pump 1 is particularly well secured against unauthorized removal even when the clamping device 14 is open, because on the circumference of the infusion pole 12 one of the clamping jaws is positioned first, then the hook 20, then the other clamping jaw, and then the back of the infusion pump 1 or the housing 2.

The main section 16, with the angled arm 18 and the hook 20 on the one hand and the securing lever 24 and the securing section 27 on the other, is designed as a flat stamped sheet metal part. This flat main section 16 is parallel to the bottom 8 shown in FIG. 1. The pivot axis of the pivot joint 17 is perpendicular to the drawing plane of FIG. 2.

The securing section 26 is latched into a latching device 28 at the released position shown in FIG. 2 and is held there, so that in particular the hook 20 cannot swing freely around when, for example, the housing 2 with the infusion pump 1 is transported from one infusion pole 12 to another.

In order to transfer the securing device or the housing 2 from the released position shown in FIGS. 1 and 2 to the state secured to the infusion pole 12, the main body 16 is pivoted counterclockwise by 900 so that the hook 20 engages behind the infusion pole 12. In the process, the securing section 26 is pivoted in the direction of the front flap 6, so that when the front flap 6 is subsequently folded down, an opening or recess 30 formed at the edge of the front flap 6 is moved over the securing section 26 in such a way that the latter engages in the recess 30 and is held in a form-fitting manner.

FIG. 3 is a section of the exemplary embodiment of the infusion pump assembly from FIG. 1 as seen from the bottom, again omitting the bottom 8 of the housing 2. The front flap 6 is shown in its closed position and the securing device or its main body 16 in the secured position. The front flap 6 is shown in a cut-away view so that it can be seen how the securing section 26 is inserted into the recess 30.

The recess 30 is formed in a region of the front flap 6, which at the same time forms a finger recess 32 for a finger of the medical personnel.

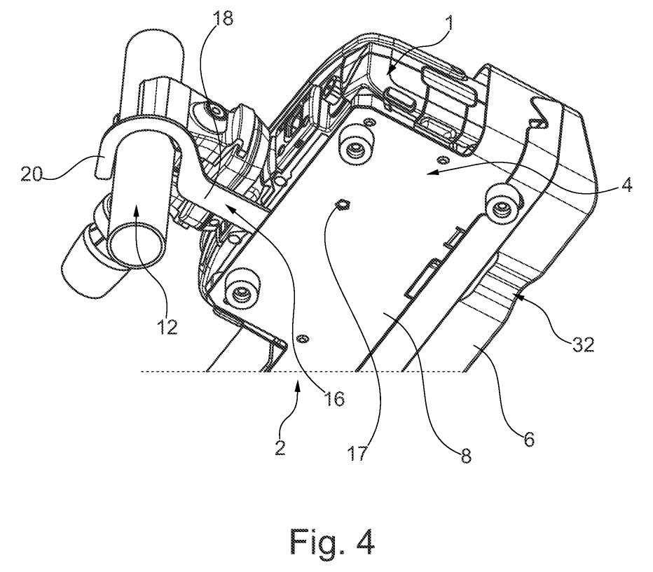

FIG. 4 is a section of the exemplary embodiment of the infusion pump assembly shown in FIG. 1, in a bottom view perspective. In this, the front flap 6 is shown in its closed position and the securing device or its main body 16 is shown in the secured position.

It can be seen that in the secured position of the main body 16, the arm 18 and the hook 20 project from the back of the housing 2, while the securing lever and the securing section are hidden.In the closed position of the front flap 6, as shown in FIGS. 3 and 4, the second (lower) edge 36 rests against the main section 4 of the housing 2.

FIG. 1shows the lock 22 used to lock the front flap 6 in this position. In this way, the engagement of the securing section 32 in the recess 30, as shown in FIG. 3, is provided so that the main body 16 cannot be pivoted about the pivot joint 17 and the hook 20 remains engaged with the infusion pole 12. This indirectly secures the securing device with the lock 22 shown in FIG. 1. This means that only one lock 22 and one (not shown) matching key are required to lock the front flap 6 and to secure the infusion pump 1 to the infusion pole 12.

### List of reference designations:

1 Infusion pump

2 Housing

4 Main section

6 Front flap

8 Bottom

10 Fastening device/clamping device

12 Infusion pole

14 Handle

16 Main body

18 Arm

20 Hook

22 Locking device/lock

24 Securing lever

26 Securing section

28 Latching device

30 Recess

32 Finger recess

36 Second (lower) edge

1. A housing for accommodating an infusion pump at least in sections, the housing being lockable and comprising:
a main section;
a flap;
a locking device; and
a securing device,
the flap being lockable with the locking device,
the locking device being configured to fix the flap in a closed position relative to the main section,
the securing device configured to secure the housing to an infusion pole,
the securing device being movable between an unsecured position and a secured position,
and the securing device being fixable in the secured position by the locking device.

2. The housing according to claim 1, wherein the securing device is indirectly lockable in the secured position by the locking device.

3. The housing according to claim 1, wherein:
the securing device comprises a main body and a pivot joint,
the main body is coupled to the housing by the pivot joint,
the main body is pivotable on the pivot joint as the securing device is moved between the unsecured position and the secured position, and
the main body has a securing section that is fixable to the flap.

4. The housing according to claim 3, further comprising a hook arranged on a region of the main body located opposite the securing section with respect to the pivot joint, the hook configured to engage behind the infusion pole.

5. The housing according to claim 4, wherein the hook is connected to the pivot joint via an arm of the main body.

6. The housing according to claim 3, wherein the main body comprises a securing lever
that extends from the pivot joint, and the securing section is formed at an end section of the securing lever.

7. The housing according to claim 3, wherein the main body is flat, and wherein the securing section is formed with the main body in a one-piece homogeneous body of unitary
construction.

8. The housing according to claim 3, wherein the securing section is configured to fix the securing device in a form-fitting manner in the secured position in a recess of the flap.

9. The housing according to claim 8, further comprising a pivot joint arranged on a first edge of the flap.

10. The housing according to claim 9, further comprising a securing lever, wherein:
the securing section is formed at an end section of the securing lever,
the securing lever extends away from the pivot joint and towards the recess of the flap in the secured position of the main body.

11. The housing according to claim 10, wherein:
the flap is coupled to the main section of the housing by the pivot joint,
the recess is formed in a region of a second edge of the flap or on the second edge of the flap, and
the recess is movable over or onto the securing section when the flap is pivoted into the closed position.

12. The housing according to claim 10, wherein:
the main body is flat and coupled to a bottom of the main section of the housing via the pivot joint,a pivot axis of the pivot joint is perpendicular to the main body and perpendicular to the bottom, andthe main body and the bottom are parallel to one another.

13. The housing according to claim 12, further comprising a latching device arranged on the bottom for the main body in the unsecured position.

14. The housing according to claim 13, wherein the securing lever extends away from the pivot joint towards the latching device in the unsecured position of the main body.

15. The housing according to claim 3, wherein a pivot angle of 45 degrees to 135 degrees is formed between the secured position and the unsecured position of the main body.
